(19) United States
(12) Patent Application Publication
(10) Pub. No.: US 2003/0054535 A1
(43) Pub. Date: Mar. 20, 2003

(54) Endoglucanase mutants and mutant hydrolytic depolymerizing enzymes and uses thereof

(76) Inventors: Michael E. Himmel, Littleton, CO (US); William S. Adney, Golden, CO (US); John O. Baker, Golden, CO (US); Todd B. Vinzant, Golden, CO (US); Steven R. Thomas, Denver, CO (US); Joshua Sakon, Fayetteville, AR (US); Stephen R. Decker, Berthoud, CO (US)

Correspondence Address: PAUL J WHITE, SENIOR COUNSEL, NATIONAL RENEWABLE ENERGY LABORATORY (NREL), 1617 COLE BOULEVARD, GOLDEN, CO, 80401-3393, US

(21) Appl. No.: 09/997,504

(22) Filed: Nov. 19, 2001

Related U.S. Application Data
(60) Provisional application No. 60/134,925, filed on May 19, 1999.

(30) Foreign Application Priority Data
May 19, 2000 (US) PCT/US00/13971

(51) Int. Cl. C12P 7/06; C12N 9/42; C07H 21/04; C12P 21/02; C12N 5/06

(52) U.S. Cl. 435/209; 435/69.1; 435/320.1; 435/325; 536/23.2; 435/161

(57) ABSTRACT

The invention provides a method for increasing the specific activity of a glycosyl hydrolase on a substrate, comprising replacing a hydrophobic surface binding amino acid of the hydrolase with a positively charged amino acid; and a method for increasing the specific activity of a glycosyl hydrolase on a substrate, comprising replacing an active site associated glycosyl-stabilizing amino acid of the hydrolase with an amino acid, the replacing amino acid not strongly retarding cellobiose from leaving the active site. The invention further provides mutant glycosyl hydrolases, which include Y245G, Y42R, and W82R.

The present application claims priority to U.S. provisional application No. 60/134,925 filed May 19, 1999, and to PCT /US00/13971, filed May, 19, 2000.

### CONTRACTUAL ORIGIN OF THE INVENTION

The United States Government has rights in this invention pursuant to Contract No. DE-AC36-99GO-10337 between the United States Department of Energy and the Midwest Research Institute.

### FIELD OF INVENTION

This invention relates to glycosyl hydrolases. More specifically, it relates to variants of Acidothermus cellulolyticus EI endoglucanase which demonstrate an increase in catalytic activity on soluble and insoluble substrates.

### DESCRIPTION OF PRIOR ART

Plant biomass, which represents the cellulosic materials that comprise cell walls of all higher plants, is the most abundant source of fermentable carbohydrates in the world. When biologically converted to fuels, such as ethanol, and various other low-value, high volume commodity products, this vast recourse can provide environmental , economic and strategic benefits on a large scale, which are unparalleled by any other sustainable recourse. See Lynd et. al., Science, 1991, 251:1318-23: Lynd et. al., Appl. Biochem. Biotechnol., 1996, 57/58:741-61. Cellulase enzymes provide a key means for achieving the tremendous benefits of biomass utilization, in the long term, because of the high sugar yeils, which are possible, and the opportunity to apply the modern tools of biotechnology to reduce costs. However, the soluble products, cellulose and glucose in particular, have been reported to be powerful inhibitors of the cellulase complex and of the individual enzyme components: endoglucanase: cellobiohydrolase: and beta-D-glucosidase. Howell, J. A.. et. al., Biotechnol. Bioeng., 1975. XVIII: 873.

The surface chemistry of acid pretreated-biomass, used in bioethanol production, is different from that found in native plant tissues, naturally digested by bacterial and fungal cellulase enzymes, in two important ways: (1) pretreatment heats the substrate past the phase-transition temperature of lignin; and (2) pretreated biomass contains less acetylated hemicellulose. Kong, F., et. al., : Appl. Biochem. Biotechnol., 1993, 34/35:23-35; Handbook on Bioethanol: Production and Utilization. edited by Wyman C. E., Washington, DC: Taylor & Francis, 1996: 424. Thus, it is believed, that the cellulose fibers of pretreated-biomass, the objective of cellulose action, are embedded in a polymer matrix different from that of naturally occurring plant tissue. Therefore, for the efficient production of ethanol from pretreated biomass, it is critical to improve the effectiveness of naturally occurring enzymes on that substrate, recognizing that nature may not have optimized mechanisms for enzymatic hydrolysis of such man-made substrates. A need therefore exits for modified cellulase enzymes which are characterized by an increase in catalytic activity on either pure, or the cellulose component in a pretreated biomass.

Cellulases are modular enzymes composed of independently folded structurally and functionally discrete domains. Typically, cellulase enzymes comprise a catalytic domain, comprised of active site residues, and one or more cellulose-binding domains, which are involved in anchoring the enzyme to cellulose surfaces. There are 21 families of catalytic domains, and each are classified on the basis of similarity of their amino acid sequences. The three-dimensional structure of 14 of those enzymes has been determined. These families exhibit a diverse range of folding patterns, but each maintains a conserved catalytic cleft. Cellulose hydrolysis is accompanied by either inversion or retention of the configuration of the anomeric carbon. Generally, for the retaining enzymes, the leaving group is the non-reducing side of the cellulose. In contrast, for inverting enzymes, the leaving group is the reducing side of the cellulose. Although the folding pattern of the catalytic domains and the precise mechanisms of hydrolysis vary, their active site features remain similar. All catalytic clefts for the cellulase enzymes include two catalytic carboxyl residues. Most glycosyl hydrolase enzymes, that depolymerize polysaccharide molecules, share these structural features in common.

Highly thermostable cellulase enzymes are secreted by the cellulolytic thermophile Acidothermus cellulolyticus. These enzymes are disclosed in U.S. Pat. Nos. 5,110,735, 5,275,944, and 5,536,655, which are incorporated by reference, as though fully set forth herein. This bacterium was isolated, in an acidic thermal pool at Yellowstone National Park, from decaying wood and is on deposit with the American Type Culture Collection under collection number: ATCC-43068. The cellulase complex produced by this organism contains several different cellulase enzymes. These enzymes are resistant to end-product-inhibition from cellobiose and are active over a broad pH range, including those pH's at which yeast's are capable of fermenting glucose to ethanol. A novel endoglucanase, known as EI, is secreted by Acidothermus cellulolyticus into the growth medium. This enzyme is generally described in U.S. Pat. No. 5,275,944: EI endoglucanase. It is described as exhibiting a specific activity of 40 micromoles glucose released from carboxymethylcellulose/min./mg protein.

Recombinant enzymes that are useful in the digestion of cellulose have been suggested for use to augment or replace costly naturally-occurring fungal cellulases. U.S. Pat. No. 5,536,655, relates to EI endoglucanase as a candidate for recombination because the gene encoding EI has been characterized, cloned, and expressed in heterologous microorganisms. A new modified EI endoglucanase enzyme has also been purified, and four peptide sequences have been isolated. These four sequences include the signal, catalytic domain (“cd”), linker, and cellulose binding (“CBD”) domains of the peptide. In SEQ ID NO: 3 of U.S. Pat. No. 5,536,655 a single 521 amino acid linear-strand peptide is described that contains the EIcd portion of the enzyme.

Information gained from the x-ray crystallographic structure of E1, Sakon, J., et al. Crystal Structureof Thermostable Family 5 Endocellulase E1 from Acidothermus cellulolyticus in Complex with Cellotetraose, Biochemistry, Vol. 35, No.33, 10648-10660, 1996, is useful in the selection of several amino acid sites, for replacement with non-native amino acids of varying chemistry. However, prior to the work of the present invention, no replacements resulting in an increase in catalytic activity have been identified.

Enhancement in the catalytic activity of EI, or glycosyl hydrolases in general, would improve the cost efficiency of a process for the conversion of pretreated biomass to ethanol. Thus, in view of the foregoing considerations, there is an apparent need for variant endoglucanases having enhanced catalytic activity on cellulose substrates. Variants in the EIcd may be generated through site-directed-mutagenesis of the EI nucleotide sequence for translation into a protein having an increase in catalytic activity over the wild-type EI.

### SUMMARY

It is a general object of the present invention to provide variant cellulase enzymes characterized by an improvement, over the wild-type enzyme, in the catalytic digestion of cellulose substrates. Another object of the invention is to increase the specific activity of EI endoglucanase on pretreated biomass substrates.

Another object of the invention is to provide a method for increasing the specific activity on an insoluble substrate of a hydrolytic depolymerizing enzyme that is a structural analog of EI endoglucanase in the sense of having a binding site for the leaving-group by replacing an active-site residue that binds strongly to the leaving group with another that binds much less strongly to the leaving group.

It is yet another object of the invention to provide a method for increasing the specific activity of a glycosyl hydrolase on a substrate by replacing an active site glycosyl-stabilizing amino acid residue with a residue that does not strongly retard cellobiose from leaving the active site.

The foregoing specific objects and advantages of the invention are illustrative of those which can be achieved by the present invention and are not intended to be exhaustive or limiting of the possible advantages which can be realized. Thus, those and other objects and advantages of the invention will be apparent from the description herein or can be learned from practicing the invention, both as embodied herein or as modified in view of any variations which may be apparent to those skilled in the art.

In some aspects, the invention provides a method for making a glygosyl hydrolase characterized by an increase in catalytic activity on an insoluble substrate, comprising replacing an active site associated glycosyl-stabilizing amino acid of the hydrolase with an amino acid, the replacing amino acid not strongly binding a disaccharide product in the active site, yet not adversely effecting enzymatic activity, and a method of making a glycosyl hydrolase characterized by an increasing catalytic activity on a soluble substrate, comprising replacing a hydrophobic surface binding amino acid of the hydrolase with a positively charged amino acid.

The invention further provides glycosyl hydrolase variants and mutants.. In some embodiments, these variants and mutants are Y245G, Y42R, or W82R. Many forms of these variants or mutants are to be included within the scope of the present invention, and may be characterized by their enhanced catalytic activity and amino-acid sequence that is not a wild-type sequence of a glycosyl hydrolase.

### BRIEF DESCRIPTION OF THE DRAWINGS

The accompanying drawing, which is incorporated in and which constitutes a part of the Specification, illustrates at least one embodiment of the invention, and together with the description, explains the principles of the invention.

FIG. 1 is a graphic representation of the Connolly surface rendering of the E1 endogluconase Y245G mutation showing, as represented by the circular white spaces, the location of the cellodextrin substrate. The figure-eight-shaped-white-space, adjacent the +2 location, represents the location where the glycine for tryptophan substitution has been made in accordance with one example of the invention.

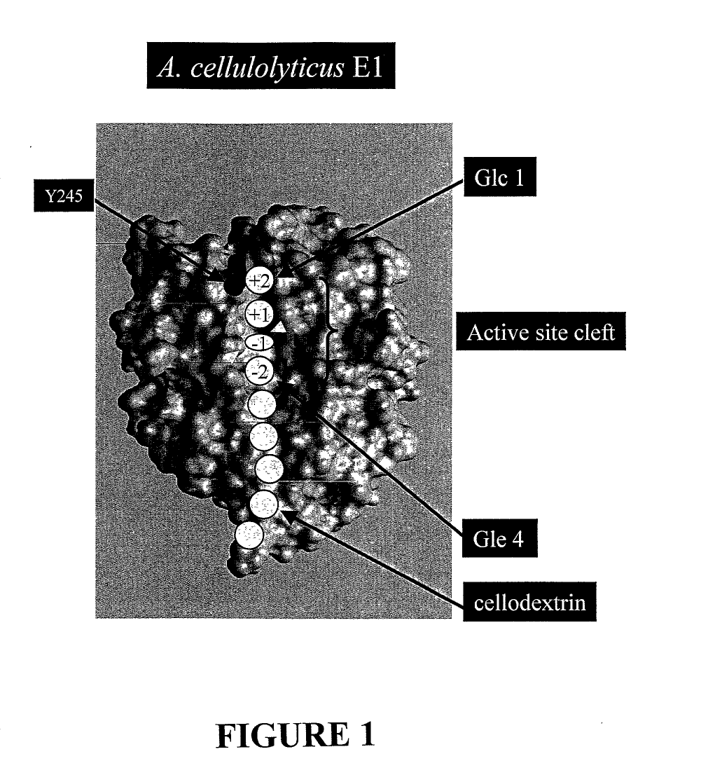

FIG. 2—Release of soluble sugars from phosphoric-acid swollen Cellulose by Wild-type and mutant Cel5A enzymes, in the presence and absence of A. niger beta-D-glucosidase. The assays were carried out at 38 degrees C., pH 5.0 in 20 mM acetate, in closed vessels. Substrate loading at 5 mg/ml; Cel5A loading sat 28 micrograms (approximately 70 nanomolar) per ml. Purified A.Niger beta-D-glucosidase (where present) was added at 45 microgram/ml. AH—CB, anhydro-cellobiose; AH-Glc; anhydro-glucose.

FIG. 3—Effect of product cellobiose concentrations on the kinetics of saccharification of PYP by wild type and Y245G mutant versions of Cel5A (assayed in combination with T. reesei Cel7A). The concentrations of cellobiose in the DSA effluent fractions (left-hand axis) are co-plotted with the saccharification progress curves (cumulative sugar released, as a percentage of that theoretically available) for the binary mixtures (1:19 molar ratio) of endoglucanase (Cel5A-wt or Cel5A-Y245G) with T. reesei Cel7A. The horizontal dashed line at 1.88 mM cellobiose represents the value of Kᵢ for inhibition of the wild-type Cel5A by cellobiose; the corresponding Kᵢ value for the mutant Y245G, at 29.7 mM, is far off the scale of the plot.

### DESCRIPTION OF THE PREFERRED EMBODIMENTS

Unless specifically defined otherwise, all technical or scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

The sequence listings herein include critical mutations that distinguish them functionally and compositionally from those amino acid sequences that are set forth in U.S. Pat. No. 5,536,655 SEQ ID NO:3. The particular sequence embodiments provided as part of the present invention are intended to include not only the specific sequence identified in the particular listing, but also any and all conservatively modified variants thereof.

“Structural analogs” means the structural analogs of E1 also benefiting from the E1 Y245G class of mutation, and include glycosyl hydrolases that provide stabilization for the leaving group, such as Van der Walls interaction, with an aromatic, sulfhyral, or hydrophobic side chain containing amino acid residues, and/or via hydrogen bonding interaction with amino acid side chains capable of hydrogen bonding to the sugar hydroxyl oxygen of hydrogen atoms. These analogous enzymes include both retaining and inverting enzymes.

Three examples for probing the possibility that the specific activity of an E1 glycosyl hydrolase can be increased, in a cellulose substrate, by site-directed mutagenesis (“SDM”), are provided. The first method describes replacing two hydrophobic surface-binding amino acid residues of the enzyme, such as residues tryptophan 42 and tyrosine 82 (See SEQ ID NO: 3 of U.S. Pat. No. 5,536,655), with a positively charged residue, such as is arginine (referenced herein as SEQ ID NO: 1 W42R; and SEQ ID NO:2 Y82R, respectively).

The second method includes replacing an active-site glycosyl-stabilizing amino acid residue of the enzyme, such as a tyrosine residue (See for example tyrosine residue 245 of SEQ ID NO: 3 in U.S. Pat. No. 5,536,655), with a residue which does not strongly retard cellobiose from leaving the active-site, such as glycine (referenced herein as SEQ ID NO:3 Y245G), alanine, valine, or serine, not strongly retarding cellobiose from leaving the active site. Glycosyl hydrolase structural analogs of E1 Y245G are set forth in Table 1. For example, in the Table, for PDB code enzyme 1 A3H (Brookhaven Data Base, Brookhaven National Laboratories), a replacement of Trp39 with Gly would remove Van der Waals stabilization of cellobiose (the reaction product),which would then not strongly bind in the active-site, in the same manner as in the replacement made according to the E1 Y245G example.

TABLE 1

| PDB code of Glycosyl | Mutation Sites | Mutation Sites: |
|---|---|---|
| Hydrolase Enzymes | E1 Tyr245 Analog | E1 Gln247 Analog |
| Structually Related to E1 | | |
| 1A3H | Trp39 | Gln180 |
| 1BQC | Trp171 | Gln169 |
| 1CEN | Trp212 | Gln16, Asp319 |
| 1CZ1 | Phe229, Phe258 | |
| 1EDG | Trp259, Trp181 | |
| 1EGZ | | Gln172, Gln173, Lys205 |
| 2MAN | Trp30 | |

Various mutagenesis kits for SDM are available to those skilled in the art and the methods for SDM are well known. Three to four mutations were made for each E1 site W42, Y82, and Y245, including Ala, Gly, Glu, and Arg. The examples below illustrate process for making and using these enzymes.

### DETAILED DESCRIPTION OF THE PREFERRED EMBODIMENTS

The QuickChange SDM kit, a trademark of StrataGene, San Diego, Calif., was used to make point mutations, switch amino acids, and delete or insert amino acids in SEQ ID NO: 1. (See SEQ ID NO: 3 of U.S. Pat. No. 5,536,655). The QuickChange SDM technique was performed using a thermo-tolerant Pfu DNA polymerase, which replicates both plasmid strands with high fidelity and without displacing the mutant oligonucleotide primers. The procedure used a polymerase chain reaction (“PCR”) to alter the cloned EI DNA (SEQ ID NO: 6 of U.S. Pat. No. 5,536,655). The basic procedure used a super-cooled, double-stranded DNA (dsDNA) vector with an insert of interest and two synthetic oligonucleotide primers containing the desired mutation. The oligonucleotide primers, each complementary to opposite strands of the vector, extend during temperature cycling by means of a Pfu DNA polymerase. On incorporation of the oligonucleotide primers, a mutated plasmid containing staggered nicks was generated. Following temperature cycling, the product was treated with the restriction enzyme, DpnI. The DpnI endonuclease (target sequence: 5′-(6-methyl)GATC-3′) was specific for methylated and hemimethylated DNA and was used to digest the parental DNA template and to select for mutation-containing, newly synthesized DNA. The nicked vector DNA, incorporating the desired mutations, was then transformed into E. coli. The small amount of starting DNA template required to perform this method, the high fidelity of the Pfu DNA polymerase, and the low cycle number all contributed to the high mutation efficiency and a decrease in the potential for random mutations during the reaction.

### EXAMPLE 1

Template DNA (pBA100) was constructed using a 2.2 kb Bam H1 fragment carrying most of the E1 gene, including its native promoter, which functions in either E. coli or S. lividans, and approximately 800 kb of upstream sequence was sub-cloned into pUC 19. The downstream Bam HI site cleaved the E1 coding sequence at a point such that the protein was genetically truncated near the beginning of the linker peptide. Thus, the construct encoded a protein, which included a signal peptide, the N-terminal cd, the entire linker region, and the first few amino acids of the C-termninal linker.

Using knowledge of the amino acid sequence of the crystalline Elcd structure, which was produced by papain cleavage of the holo-E1 protein, two different tandem translation terminator codons were introduced into the coding sequence in frame with the last amino acids present in the EIcd crystal structure. The 2.2 kb Bam HI fragment, named pBA100, in pUC19 containing the tandem stop codons served as the template for the following mutagenesis reactions.

The three target sites of SEQ ID NO: 3 of U.S. Pat. No. 5,536,655 selected for mutagenesis were W42, Y82, and Y245. Four or five pairs of mutagenic oligonucleotides were designed for each target site, such that 4 or 5 different amino acid substitutions would be created at each of the target sites. Both strands of the template molecule were copied and mutagenized during the in vitro DNA synthesis reaction using the QuickChange In Vitro Mutagenesis kit (Strata Gene, San Diego, Calif.). The two mutagenic oligonucleotides were completely complementary to each other, but differed by one or more nucleotide from the template DNA strands. Each mutagenic oligonucleotide was designed such that the nucleotides to be changed were located near the center of the oligonucleotide sequence, with approximately equal lengths of complementary sequence stretching out in both the 5′ and 3′ directions from the site of mutagenesis. Typically, mutagenic oligonucleotides were 26-30 nucleotides in length, but were sometimes longer due to considerations surrounding the melting temperature (“Tₘ”). The Tₘ was critical in the design of the mutagenic oligonucleotides because the oligonucleotides used in mutagenesis reactions required a Tₘ at least 10 degrees C. higher than the temperature for the DNA synthesis reaction (68 degrees C.). Accordingly, the effective mutagenic oligonucleotides required a Tₘ of at least 78 degrees C.

Template DNA from E. coli XL1-blue cells transformed with Dpnl treated mutagenized-DNA, was prepared for sequencing using the QIAprep-spin plasmid purification mini-prep procedure, provided by Quagen, Inc. The transformed XL1-blue cells were grown over-night in 5 mL of LB broth with 100 microgram/mL ampicillin. Cells were separated by centrifugation and the plasmid was isolated. Presence of the 2.2 kB insert was confirmed by digestion with BamH1, followed by agarose electrophoresis. Transformants having insert containing DNA were precipitated in ethanol and then PEG. The DNA template concentration was adjusted to 0.25 microgram/microliter, and the DNA was sequenced using procedures well known in the art.

Transformed E. coli XL1/blue cells were cultured over-night at 37 degrees C. on LB plates containing 100 microgram/mL ampicillin. A single colony was then used to inoculate 200 mL of LB broth containing 100 microgram/mL ampicillin in a 500 mL baffled Erlenmeyer flask. This organism was grown in a reciprocating incubator at 250 rpm, for 16-20 hours, at 37 degrees C. This culture was used to inoculate a 10L BioFlow 3000 Chemostat, New Brunswick Scientific, New Brunswick N.J. The culture medium comprised LB broth, 100 microgram/mL ampicillin, and 2.5% filter sterilized glucose. The pH, temperature, agitation rate, and dissolved oxygen parameters were maintained throughout the fermentation. The pH was controlled at 6.8 using a 2M potassium hydroxide solution. Temperature was controlled at 30 degrees C. in order to prevent the formation of inclusion bodies. The agitation rate was 250 RPM. The dissolved oxygen polarographic probe was calibrated using nitrogen (0% activity=4.0 L/min.) and house air (100% activity at 4.0 L/min). An oxygen and air mixture was used to maintain the dissolved oxygen tension at 20%. The cells were cultured 24-28 hours, which typically resulted in a maxim optical density of between 15-20. The cells were then harvested in a continuous centrifuge at 25,000 rpm.

Fifty grams of cells (wet/wt.) were added to the chamber of a stainless steel bead beater containing 200 g of 0.1 mm glass beads, and 200 mL of 20 mM Tris, pH 8.0, buffer. Cell lysis was carried out for 5 min. in the bead-beater, while the chamber was chilled with ice. The contents of chamber was diluted two-fold with buffer and divided into centrifuge bottles (250 mL). The cell debris was removed by centrifugation at 13,000 rpm, 4 degrees C., for 25 min. The supernatant was decanted, the pellet suspended in buffer, and the cells were milled and separated by centrifugation.

Two procedures were used in the initial purification of the enzyme(s). In the first, the supernatants were pooled and brought to 0.5M (NH₄)₂SO₄. The supernatant was divided, into 250 mL centrifuge bottles, and heated in a 65 degree C. water bath, for 50 min., in order to denature non- EI (i.e., E. coli) protein. Precipitated proteins were separated at 4 degrees C. by centrifugation at 13,000 rpm, for 25 min. The supernatant was then filtered, through a glass fiber filter pad, prior to the chromatography step. An improved purification procedure resulted in a substantial reduction in the overall processing-time, but retained an equivalent yield of protein. This procedure involved lysing the cells using the mill, combining the supernatants, and diluting the combined supernatant with 20 mM Tris, pH 8.0, buffer until the conductivity of the supernatant was less than 2000 microS/cm. The resulting material was separated with an expanded-bed-adsorption-chromatography system using DEAE packing in a Pharmacia Streamline column.

Two methods were developed for the subsequent purification of the mutant EI enzymes from the E. coli XL1/blue cell lysates described above. The original protocol involved a substantial amount of sample preparation prior to purification. An improved procedure was subsequently developed using new chromatography resins which eliminated the need for much of the sample preparation and clarification of the cell lysate.

The original purification protocol consisted of the following steps. The cell lysate, which contained 0.5 M (NH₄)₂SO₄, was loaded on a Pharmacia preparative which had been packed with a 500 mL bed volume of Pharmacia Fast Flow, low substitution Phenyl Sepharose media. A Pharmacia BioPilot system was used to control chromatography. After the cell lysate was loaded, the column was washed with three to five volumes of 20 mM Tris, pH 8.0, buffer containing 0.5 M (NH₄)₂SO₄, at a flow rate of 0.50 DL/min, after which the recombinant EI enzyme(s) (“rEI”) was eluted with 3.2 column volumes, descending linear gradient, to zero-percent salt of 20 mM Tris, pH 8.0, buffer. The rEI eluted in fractions resulting from approximately zero percent salt. These fractions were combined, and dialyzed against 20 mM Tris, pH 8.0, buffer for 12 hours. The dialyzed-concentrated-protein was subjected to anion-exchange-chromatography in a Pharmacia Q-sepharose HiLoad 16/10 high performance column. The enzyme was loaded in 20 mM Tris, pH 8.0, buffer, and was eluted by a shallow linear gradient (22 column volumes) using the same buffer with 0.5 M NaCl. Most of the rE1 mutant enzyme(s) eluted at 150 mM NaCl. The active fractions were then combined, concentrated, and loaded in a Pharmacia Superdex 200 HiLoad prep grade column at a 0.5 mL/min. flow rate in 20 mM acetate, pH 5.0, buffer with 100 mM NaCl. The rEI enzymes eluted as a single-symmetrical-peak which is indicative of a highly homogenous compound. The purity of the rEI enzyme(s) was confirmed with SDS-PAGE using Novex pre-cast 8-15% gradient gels, and contained a single 40 kDa band. The protein concentrations were then determined based on absorbance at 280 nm using a molar extinction coefficient which had been calculated for each individual replacement amino acid.

The improved method eliminated the need for clarification of the supernatant after lysing the cells. The cell lysate, which had been adjusted to a conductivity of less than 2000 micro S/cm, was loaded directly onto a Pharmacia Streamline column packed with Streamline DEAE (a weak anion-exchanger) fluidized at a flow rate of 15 mL/min with 20 mM Tris, pH 8.0, buffer. After the column matrix was washed free of the cell debris, and the UV absorbance returned close to zero, the flow was reversed to a down-flow orientation and the proteins were eluted using a linear gradient of 20 mM Tris, 1M NaCl, pH 8.0, buffer. Active fractions were pooled, and ammonium sulfate was added to a final concentration of 0.5M. These samples were then loaded on a Phenyl Sepharose HiLoad column. After the column was washed, with 3-5 column volumes of the starting buffer, the rEI enzyme(s) was eluted, by a 3.2 column-volume descending linear gradient, to zero percent salt in 20 mM Tris, pH 8.0, buffer. The final purification step and buffer exchange was made using a Superdex 200, HiLoad prep-grade-column with a flow rate of 0.5 mL/min., in 20 mM acetate, pH 5.0, buffer with 100 mM NaCl. Mutant rEI enzymes eluted as single symmetrical peaks indicating a high level of homogeneity. The protein concentrations were then determined as described above.

Solid-phase, immunology methods were used to detect the expressed enzyme. Imunoblots and Western blots were used to verify the presence of EI and EI mutant enzymes. For immunoblots, 2 microliters of a chromatography sample fraction was applied to nitrocellulose and allowed to air dry. For Western blots, 3-5 micrograms of protein was added to each lane and the proteins were subjected to electrophoreses. A monoclonal antibody specific for EI was then added after the proteins had been blotted to the nitrocellulose. This was followed by the addition of a goat anti-mouse-IgG alkaline phosphate-labeled antibody. Bound EI was visualized by the precipitation of the substrate.

The Michaelis constant (“Kₘ”), and maximal rate (“Vₘₐₓ”) for each enzyme preparation were determined from the rates of cellobiose production, at different cellotriose concentrations. Replicate assay mixtures containing 5 mM acetate buffer, pH 5.0, 10 g/mL BSA, and cellotriose ranging from 0.0793 mM (0.04 mg/mL) to 1.9825 mM (1.0 mg/mL) were prepared. Each assay mixture was pre-incubated at 50 degrees C. for 10 min. prior to the addition of 0.00272 micromolar (0. 1092 microgram/mL) enzyme, which was also made up in 5 mM acetate buffer with 10 microgram/mL BSA. The final assay volume was 0.1 mL.

At set-time intervals, an aliquot of the reaction mixture was pulled and immediately analyzed for the release of cellobiose using a Dionex DX300 chromatography system and a Dionex PAD2 pulsed amperometric detector having a gold working electrode. The response of this detector was optimized for the detection of carbohydrates using a waveform defined by the following time and potential settings: t₁=420 msec.; EI=+0.05 V; t₂=180 msec.; E₂=+0.75 V; t₃=360 msec.; and E₃=−0.15 V. Separation of the reaction products, from the substrate, was achieved on a Dionex CarboPac PA-1 analytical (4×250 mm) column equipped with CarboPac PA-1 (4×50 uard column, 500 mM sodium hydroxide eluent, and a flow rate of 1.5 mL/min. The amount of cellobiose present for each time-point-sample was quantified by comparing the area of the cellobiose peak against a linear calibration curve. The kinetic constants were determined with a double-reciprocal-plot, where the reciprocal of the rate of cellobiose produced was plotted as a function of the inverse of the substrate concentration. This resulted in a straight line function having an intercept of 1/Vₘₐₓ and a slope of Kₘ/Vₘₐₓ.

All diafiltration saccharification assays (“DSA”) (those that provided the original discovery of enhanced activity in the Y245G mutant) were carried out at pH 5.0 in sodium acetate buffer containing 0.02% sodium azide. Substrate loading, for each assay, comprised 104 mg (dry wt.) of pretreated-yellow-poplar (“PYP”). This weight was equal to a load having 4.7% biomass and a 3.2% cellulose. The substrate was ground to a maximum particle size of between 10 and 500 microns. In the initial assays of the present study (those that first revealed the enhanced activity of the Y245G mutant with respect to that of the wild-type) selected enzymes, such as the wild-type or mutant A. cellulolyticus E1 catalytic domain, were loaded at 56.4 nanomoles enzyme/g cellulose and were carried out at 50° C. Each assay mixture further included 487 nanomoles of T reesei cellobiohydrolase (CBH 1) enzyme/g cellulose, which resulted in an enzymatic solution of 10% endoglucanase and 90% cellobiohydrolase, which resulted in an enzymatic solution of 10% endoglucanase and 90% cellobiohydrolase. The endoglucanase proportion in the mixture was high enough to provide a readily-measurable activity, but was sufficiently below an optimal endoglucanase concentration, which causes sugar release and synergism to make the results highly sensitive to differences in endoglucanase activity. Later diafiltration saccharification assays (those used to delineate the exact manner in which the enzyme activity of the Y245G mutant was enhanced with respect to that of the wild-type) were carried out at 38° C., with a given endoglucanase loaded at a ratio of 1:19, or 5% to 95%, to the cellobiohydrolase (Cel7A), at a total enzyme loading equal to 75% of that used in the initial studies.

The temperature optimum for maximum activity was determined for each EI mutant using p-nitrophenol-beta-D-cellobioside as the substrate in a 20 mM acetate, 100 mM NaCl, pH 5.0, buffer. Equivalent concentrations of enzyme were used (0.4 microgram/mL) in a 30 min. assay at various temperatures. After a 30 min. incubation period, the reactions were stopped with the addition of 2 mL 1M NaCO₃ and the amount of p-nitrophenolate anion released was measured by absorbance at 410 nm. The temperature optimafor the mutants claimed was found to be essentially identical to that of the native EI.

While the PCR technique is well known in the art and commonly performed with reagents packaged in kit form, the following modifications provided nucleotide substitutions at all targeted sites, which are identified in the Table 2 below. Good annealing of the DNA template and primers was critical. The Tₘ for this process was a function of the length of the oligonucleotide, the concentration of monovalent cations, and the GC content of the oligonucleotide. The Tₘ was calculated according to the formula: Tₘ=81.5+16.6(log[Na+]) +0.41 (% G+C)−(675/N)−% mismatch, where N is the primer length in base pairs, and [Na+] is the sodium ion concentration. The Tₘ increased with an increase in the GC content, salt concentration, and oligonucleotide length. Because the EI sequence is very GC-rich (62.8%), relatively short mutagenic oligonucleotides were used (i.e., 26-30 bases). However, in some situations because of the relatively AT-rich segment of DNA around a site (i.e., lower Tₘ), such as was the case for the Y82 mutations, longer mutagenic oligonucleotides (38 bases) were synthesized in order to obtain an oligonucleotide having a suitably high Tₘ. The following Table 2 illustrates the mutations in SEQ ID NO:6 U.S. Pat. No. 5,536,655 which translated into the rEI enzymes demonstrating an increase in activity over the native protein of SEQ ID NO:3 U.S. Pat. No. 5,536,655. Changing the codons to reflect alanine , valine, or serine replacement can be made in the similar manner, and the codons for these amino acids are well known.

TABLE 2

| | Insert DNA Sequence |
|---|---|
| EI Mutation Target Site | From PCR Mutation |
| SEQ ID NO:3 | SEQ ID NO:6 |
| U.S. PAT. NO. 5,536,655 | U.S. PAT. NO. 5,536,655 |
| EIW42 NATIVE | GTGCACGGTC TCTGGTCACG |
| | CGACTACCG |
| EIW42R | GTGCACGGTC TCCGGTCACG |
| | CGACTACCG |
| EIY82R NATIVE | GC CGAACAGCAT CAATTTTTAC |
| | CAGATGAATC AGGACC |
| EIY82R | GC CGAACAGCAT CAATTTTCGC |
| | CAGATGAATC AGGACC |
| EIY245G NATIVE | CGCGACGAGC GTCTACCCGC |
| | AGACGTGG |
| EIY245G | CGCGACGAGC GTCGGCCCGC |
| | AGACGTGG |

### EXAMPLE 2

### Mutant EI and Native EI cd

The present example is provided to demonstrate the industrial utility of the mutant EI enzymes and one native EI cd. These were purified using the purification methods described above. Purification of the mutant enzymes destined for kinetic analysis was necessary because any precise comparison of specific activity required knowledge of the enzyme(s) concentration. For this reason, considering the specific change in the amino acid compositions made a determination of the molar extinction coefficients of the recombinant enzymes. Although all active mutant EI enzymes behaved similarly during purification, some mutant enzymes showed a substantial departure from the EI cd behavior on anion exchange chromatography. All transformed strains of E. coli examined were found to produce adequate levels of mutant EI enzymes (i.e., approximately 0.5 to 1 mg/10 L culture).

Ten-Liter cultures of the transformed E. coli expressing active enzymes were grown and each mutant enzyme was purified to homogeneity using an improved three-step column chromatographic method. The purified rEI endoglucanase enzymes (including the EI control) were characterized for activity on cellotriose and PYP.

Michaelis-Menten kinetics of the mutant EI enzymes and the native enzyme were determined. As a result, it was concluded that the W42R (SEQ ID NO:1) and Y82R(SEQ ID NO:2) amino acid substitutions at sites W42 and Y82 of U.S. Pat. No. 5,536,655 SEQ ID No.: 3 improved the catalytic activity for this soluble substrate.

Cellotriose kinetics for the EI mutations are show in the Table 3 below. In the case of cellotriose hydrolysis, mutations which increased Kₘ (indicating probable decreases in strength of substrate binding), also displayed an increases in velocity. Thus, the arginine substitutions at sites W42 and Y82 resulted in the highest Vₘₐₓ values observed, about 15% and 75% higher than that of the native enzyme, respectively.

TABLE 3

| Enzyme/Mutant | Km (mM) | Vmax (uM/min.) |
|---|---|---|
| EI NATIVE | 0.35 | 0.86 |
| EIW42R | 0.61 | 0.99 |
| EIY82R | 0.69 | 1.5 |
| EIY245G | 0.48 | 0.85 |

These mutant EI enzymes were also tested for activity on pretreated yellow poplar using the diafiltration saccharification assay (Baker, J. O., et al, Use of a New Membrane-Reactor Saccharification Assay to Evaluate the Performance of Cellulases under Simulated SSF Conditions, Applied Biochemistry and Bioengineering, 1997, 63-65:585-595). This assay tested the ability of the modified E1 enzymes to hydrolyze an insoluble substrate in combination with T reesei cellobiohydrolase (CBH 1). This test has the advantage of taking cellulose hydrolysis to the 90% level, under conditions consistent with simultaneous saccharification fermentation, which is desirable for the use of the enzymes according to the examples provided herein. Ten-L cultures of the transformed E coli expressing active enzymes were grown and each mutant enzyme was purified to homogeneity using an improved three-step column chromatographic method. The purified EI endoglucanase enzymes (including the EI control) underwent DSA on cellulose. In Table 4, the results for the EI mutations, having at least native activity, are shown.

TABLE 4

| ENZYME/MUTANT | % SACCHARIFICATION OF PYP/96 HOURS |
|---|---|
| EI NATIVE | 44.5 |
| W42R | 46 |
| Y82R | 45.3 |
| Y245A | 50.5 |

Although 3 to 4 mutations were found for each EI site W42, Y82, and Y245, including Ala, Gly, Glu, Gln, and Arg, only three variants demonstrated no loss in native activity on insoluble substrates relative to the native enzyme. These EI variants were identified as W42R, Y82R, and Y245G. Only the EI Y245G (U.S. Pat. No. 5,536,655, SEQ ID NO:3) variant showed a significantly greater catalytic activity over native EI. DSA testing revealed that the glycine mutant enzyme (Y245G) demonstrates a 12% (+/−) 1.0%) improvement in DSA catalytic activity. This increase is explained by a decrease in cellobiose binding, and thus cellobiose end-product-inhibition at site Y245. To confirm this result, a second preparation of EI Y245G was produced from the transformed E. coli stock. This mutant EI also showed substantial increase in DSA activity over the native enzyme. i.e., 9.5% (+/− 1.0%).

Results suggesting that the relief of inhibition by cellobiose is a factor in enhanced biomass hydrolysis, with the EI Y245G mutant, are supported from the following observations: (1) addition to the DSA enzyme cocktail of sufficient beta-D-glucosidase, to reduce the cellobiose concentration the assay reactor below the level of HPLC select ability, has the effect of abolishing most of the difference in performance between native and mutant EI; and (2) Ki values for inhibition of hydrolysis of 4-beta-D-cellobioside (MUC) by native and mutant EI indicate that the mutant catalytic domain binds cellobiose 15 times less tightly than does the native enzyme, i.e., an increase in Ki from 2 to 30 mM cellobiose. The decrease in apparent binding energy is 1.7 kcal/mol.

### EXAMPLE 3

### Inhibition Constants for Cellobiose with Wild-Type and Y245G Mutant

The present example is provided to demonstrate the utility of the invention for enhancing the catalytic activity of cellulases over wild-type non-mutant counterpart enzymes, for use in either simultaneous saccharification and fermentation (SSF) or sequential (separate) hydrolysis and fermentation (SHF) processes.

Inhibition constants (Kᵢ) for the inhibition of the hydrolysis of 4-methylumbelliferyl-β-D-cellobioside (Sigma Chemical Co., St. Louis, Mo.) were determined under conditions matching those of the DSA and closed-tube (PASC) experiments. The enzymes (0.682 ng) were incubated for 30 min with the substrate at each of two concentrations (4 and 20 μM), in the presence of D-cellobiose (Sigma, St. Louis) )at concentrations ranging from 0 to 5 mM for the wild type catalytic domain, and from 0 to 50 mM for the Y245G mutant. At the end of the incubation period, the reaction in each 1-mL assay mixture was terminated by addition of 2 mL of 0.5 M sodium carbonate, pH 10.0. The extent of hydrolysis was then determined from the fluorescence of the ionized product, 4-methylumbelliferone, as measured in a SPEX FLUOROLOG spectrofluorometer with excitation wavelength at 380 nm and emission wavelength at 455 nm. These studies established that under these conditions, 1% or less of the in 30 min. Inhibition constants were then determined by means of Dixon plots of reciprocal velocity versus inhibitor concentration (Segel, 1975).

The mutant Cel5A enzyme, Y245G, was generated by PCR mutagenesis, and the mutant and wild type Cel5A catalytic domains were purified using the purification methods described above. Purification of the native and mutant enzymes destined for kinetic analysis was crucial for this study, because specific activities must be compared on the most precise basis possible. For this reason, the molar extinction coefficients of the recombinant enzymes were calculated by considering the specific change in amino acid composition.

Analysis of the X-ray crystallographic structure of the wild-type enzyme suggested that removal of the glucosyl-binding platform provided by Tyr-245 would substantially decrease the affinity of the leaving-group binding site for cellobiose. The results of initial-velocity kinetic experiments have shown that mutation of Tyr-245 to glycine does indeed produce a large (more than 15-fold) decrease in affinity of the active site for cellobiose, in that the Kᵢ for inhibition of the hydrolysis of 4-methylumbelliferyl-β-cellobioside (MUC) by the wild type enzyme is 1.88±0.16 mM, but is increased by more than 15-fold, 29.7±3.8 mM, for the Y245G mutant. This increase in the value of Kᵢ indicates a reduction in Cel5A/cellobiose binding energy on the order of 1.5 kcal/mol.

### EXAMPLE 4

### Membrane-Reactor Assays of Activity Versus Biomass Cellulose

The present example is provided to demonstrate the utility of the invention for enhancing the catalytic activity of cellulases over wild-type non-mutant counterpart enzymes for use in simultaneous saccharification and fermentation (SSF). The PASC-saccharification experiments discussed above may be considered to mimic, in a limited way, one industrial application of cellulase enzymes, namely the saccharification step of separate saccharification and fermentation (SHF), which is one possible configuration for the process of converting biomass cellulose to fuel ethanol, or other chemical products. Although a highly processed pure cellulose such as PASC is unlikely to be chosen as an industrial feedstock, and most industrial applications involving substantial conversion of more complex and less processed feedstocks will almost certainly use a complex of enzymes rather than one, the PASC experiments do resemble SHF in that the cellulose depolymerization is carried out in a closed system, so that products accumulate to substantial concentrations. The discussion of the Y245G mutant endoglucanase and its catalytic performance will now be concluded with the example of additional experiments that involve conditions mimicking those encountered in the processing of an actual candidate cellulosic feedstock in a somewhat different industrial application, namely simultaneous saccharification and fermentation, or SSF.

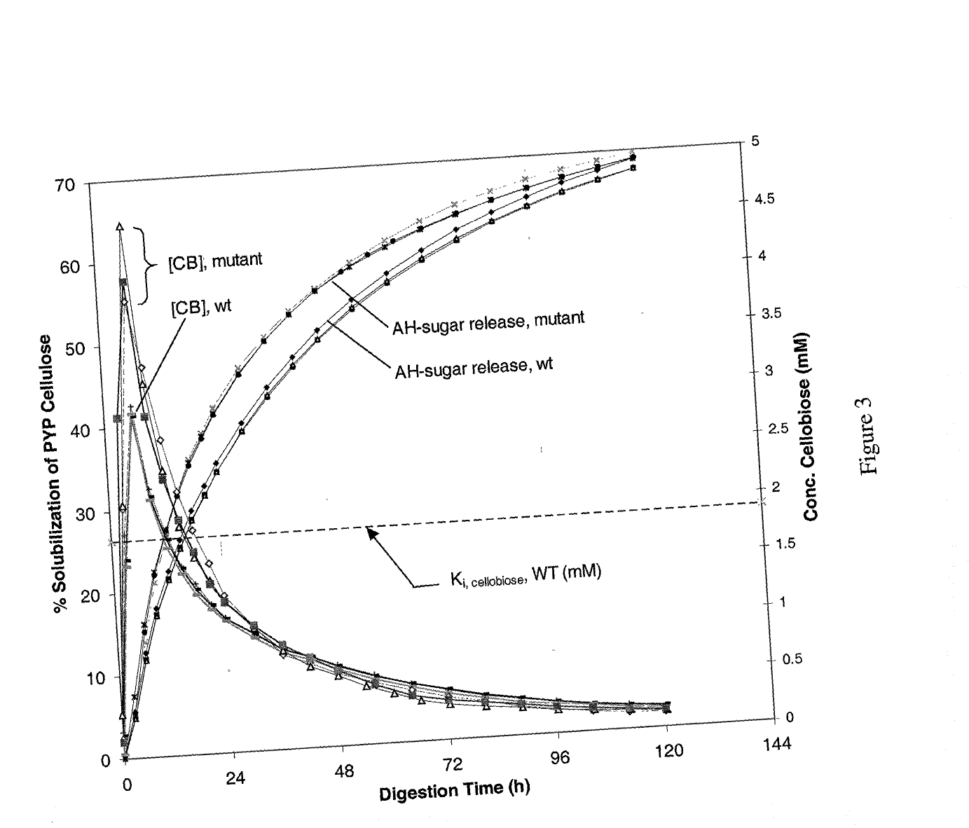

The DSA progress curves presented in FIG. 3 illustrate the enzymatic saccharification of the cellulose component of dilute-acid-pretreated yellow poplar (PYP), which is poplar sawdust from which most of the hemicellulosic material, and some of the lignin, has been removed by dilute-acid hydrolysis at high temperature. Left behind in the PYP substrate is a sterically complex mechanical intermixture of predominantly cellulose (approx. 58%) and lignin (approx. 35%), arranged in a matrix retaining substantial elements of the original wood structure. Attack on this physically and chemically heterogeneous substrate is carried out (FIG. 3) by binary mixtures of either wild-type or Y245G-mutant Cel5A endoglucanase, used in each case with T. reesei cellobiohydrolase-I (Cel7A). In nature, and in virtually any industrial process involving substantial enzymatic saccharification of biomass material, the effective digestion of cellulose is carried out not by one enzyme, but by mixtures of cellulolytic enzymes acting synergistically (Baker et al., 1995; Nidetzky et al., 1994). In the experiments shown in FIG. 3, the binary mixture of one endoglucanase (Cel5A wild-type or the Y245G mutant) with one exoglucanase (Cel7A) can be regarded as a minimal effective system for attack on an insoluble, and still significantly crystalline, cellulosic material. In earlier studies of the depolymerization of microcrystalline cellulose by binary mixtures of purified cellulases obtained from various organisms, the Cel5A/Cel7A pair was both the most active and the most synergistic of the pairs tested. Making Cel5A the minority component in the current (5:95 molar ratio) assay mixtures serves to make the resultant activities very sensitive to differences in Cel5A activity.

The digestions of FIG. 3 were carried out in a stirred membrane reactor that was constantly swept by a buffer flux through the membrane. In this diafiltration saccharification assay (DSA) (Baker et al., 1997) reactor design, macromolecular enzymes and the insoluble substrate are retained in the reactor by the membrane (an ultrafiltration membrane with nominal MW-cut-off of 5,000 kDa). The small-molecular-weight solubilized sugars, meanwhile, are continually swept out of the reactor by the buffer flux, which is then collected in timed fractions and analyzed for sugar content to provide the cumulative sugar-production progress curves shown in FIG. 3. In this assay, the removal of the solubilized sugars by the buffer flux mimics the consumption of sugars by fermentative organisms in SSF. In both cases (DSA and SSF) continuous removal of sugars greatly reduces product-inhibition of the cellulases by driving the pseudo-steady-state concentration of the sugars to a much lower level than would be present, were the sugars allowed to accumulate without removal, but does not reduce the sugar concentrations to zero. This last point will be revisited later in the discussion.

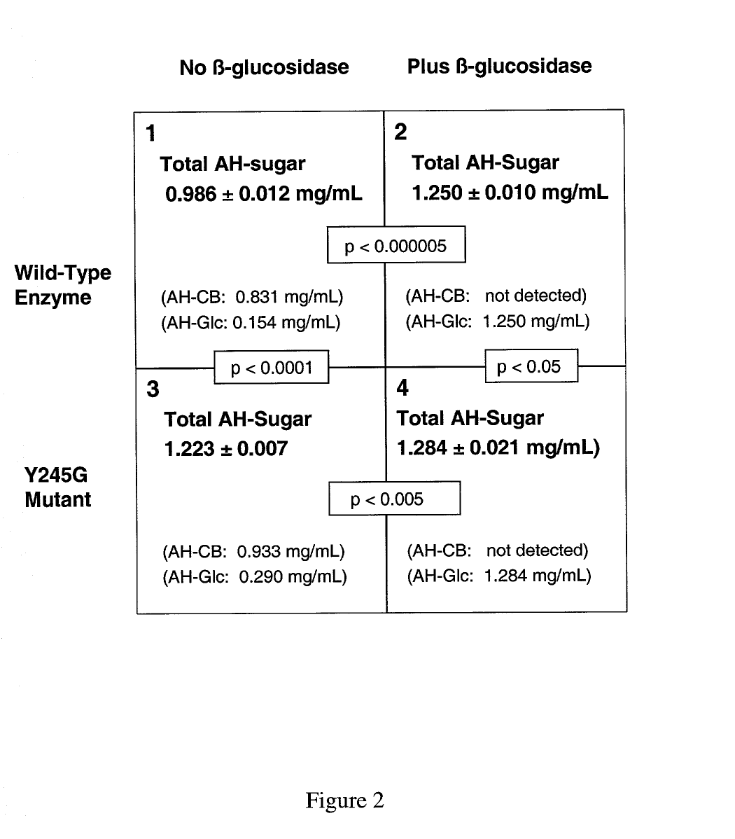

The progress curves of FIG. 3 show enhanced initial kinetics for the hydrolysis of the cellulose component of PYP by the binary enzyme mixture of the Y245G mutant and Cel7A, relative to the performance of an otherwise-identical mixture formulated using the wild-type endoglucanase. The progress curve for the enzyme mixture containing the mutant has a steeper slope over the first 24 h of the digestion, but the difference in relative rates decreases over the course of the digestion, so that by the time interval from 24 to30 h, the slope of the progress curve for the mutant-containing mixture is actually slightly smaller than the slope of the curve for the mixture with wild-type endoglucanase. From this point on, at the same time-points, the wild-type mixture will be releasing sugars more rapidly than the mutant mixture. By 120 h digestion, the wild-type mixture has essentially caught up in terms of cumulative sugar release, with the mutant mixture showing less than 2% more sugar production than the wild-type mixture. This “hare and tortoise” pattern can be explained in terms of two principal factors. First, the pseudo-steady-state concentration of cellobiose in the reaction chamber reaches its highest values in the early stages of the reaction, when the rate of production of cellobiose is higher, relative to the constant dilution rate, than later in the reaction. Plots of the cellobiose concentrations in effluent fractions are overlaid on the progress curves of FIG. 2, with the values indicated by the left-hand axis. Given that, as shown by the data in Table 2 for closed-tube digestion of PASC, most of the kinetic advantage of the Y245G mutant can be traced to relief of inhibition by cellobiose, it is not especially surprising that in these continuously-monitored experiments, the mutant mixture is seen to gain most of its advantage in the (early) stages of the digestion, when the cellobiose level, and therefore cellobiose inhibition of the wild-type enzyme, is higher. Second, in addition to the differential effect of cellobiose accumulation on the two endoglucanases, the fact that the substrate is both physically and chemically heterogeneous, with the more readily-digested material being solubilized first, means that at any given time during the digestion as shown here, the enzyme mixture with the highest early rate (in this case the Y245G-containing mixture) will be encountering more resistant material, on the average, than is the wild-type mixture, which has more of the more easily digested material remaining. In the later stages of the digestion, when cellobiose levels are much lower than earlier, the advantage of the mutant in terms of resistance to inhibition is greatly diminished, and is more than compensated for by the greater average digestibility of the material facing the wild-type enzyme, allowing the wild-type enzyme to catch up.

The fact that a single point-mutation in a single enzyme has such a clear effect in this case, even though the mutated enzyme is the minority component (5% on a molar basis) in the enzyme mixture, is very probably related to the synergistic action of endoglucanases and exoglucanases in the depolymerization of insoluble cellulose. Endoglucanases (such as Cel5A) are capable of random attack on interior glycosidic bonds of cellulose chains, which attacks create new chain-ends. The new chain-ends then serve as points of attack by exoglucanases (such as Cel7A, which is specific for reducing ends of the chains), which then act possessively to release successive cellobiosyl residues from the chains. In addition to being able to release soluble sugars from cellulose themselves, by successive attacks, the endoglucanases thus play an important role by potentiating the action of the exoglucanases.

Careful attention to the cellobiose concentrations plotted in FIG. 3 reveals that the cellobiose concentrations in the effluent fractions, even those near the peak of cellobiose concentration, do not appear to be overwhelmingly large with respect to the Kᵢ value for inhibition of the wild-type enzyme by cellobiose (Kᵢ=1.88 mM) In fact, in the effluent fractions collected between 9 and 12 hours for all six assays, the cellobiose concentration has fallen to the neighborhood of Kᵢ, and in fractions collected later the concentrations are equal to diminishing fractional values of Kᵢ. While this does not mean that one would expect no inhibition of the wild-type enzyme at these lower concentrations, it does suggest that the extent of digestion would (depending on the strength of the competing interactions of enzyme and substrate) range from moderate to small. A question is then raised by this finding in comparison with the observed strong effect of the mutation in Cel5A upon the overall activity, which effect we attribute to relief of substrate inhibition. A quite likely answer is to be found in the porous structure of the wood-derived substrate. Substantial saccharification of the biomass cellulose will require the diffusion of the enzymes into the wood-particle structure. Hydrolysis of cellulose chains inside the pores of the substrate will result in relatively high concentrations of products inside the pores, because the residual structure of the substrate particles (composed to an increasingly large extent of lignin) will provide a physical barrier to the free diffusion of products. While the cellobiose concentrations in the effluent fractions are an excellent measure of the cellobiose concentrations in the bulk fluid in the reaction chamber at the time the effluent passed through the membrane, these concentrations can only indicate probable general trends in the concentrations of cellobiose inside the pores of the substrate. The concentration of product inside the pores are probably always significantly higher than the concentrations found in the bulk solution between the particles (and reported by the effluent concentrations).

A variety of approaches may be used to generate quantitative estimates of the extent to which the mutation of tyrosine-245 to glycine accelerates the action of the binary enzyme mixture used in FIG. 3. One of the simplest, although not the most meaningful, approaches is to compare the amounts of cellulose solubilized by the two mixtures by a specific time of digestion. Using this approach, we find that the ratio of cellulose solubilized by the mutant-containing mixture, to that solubilized by the wild-type mixture, is maximal at 9 h of digestion, the mutant mixture having converted more cellulose than the wild-type by a factor of 1.25. While this “equal-digestion-time” approach is straightforward, and the only approach practical for single-end-point assays such as the closed-tube PASC assays of FIG. 2, the continuous monitoring of the assays in FIG. 3 allows one to make a more meaningful determination of relative rates. In this latter method, the continuous progress curves are used to generate estimates of the time required for the two enzyme mixtures to accomplish the same extent of conversion of the substrate. The reciprocals of these “times to target” are then used as measures of relative activities for the two mixtures. For example, an enzyme or enzyme mixture that converts 30% of the substrate in one hour is regarded as having twice the activity of an enzyme or mixture that accomplishes the same thing in two hours, or twice the time. This approach is especially attractive in dealing with substantial conversion of heterogeneous substrates such as PYP, because, even though the nature of the substrate changes over the course of a substantial conversion, it is not unreasonable to assume that two enzymes or mixtures that convert the substrate to the same extent will have acted upon substrate of essentially the same nature over the course of the reaction. Using the “reciprocal time to target” as an estimator of relative activity, we find that the ratio of rates is maximal when a value near 35% is chosen as the target extent of digestion. The mutant-containing mixture is found to reach 35% conversion in 17.7±0.3 h (average of triplicate determinations), whereas the enzyme mixture containing the wild-type endoglucanase requires 24.7±0.2 h to accomplish the same extent of conversion. These digestion times correspond to the reciprocals 0.0405±0.0004 h⁻¹ (wild-type) and 0.0565±0.0005 h⁻¹ (mutant). The difference between these two means is significant at the p<0.0001 level, and the ratio of the means (Mutant/WT) is 1.396, indicating that in this substantial conversion of the cellulose content of a realistic industrial biomass feedstock, the mixture containing the mutant endoglucanase exhibits almost 40% greater activity than the mixture utilizing the wild-type endoglucanase.

The data previously shown in Table 3 were also collected using the diafiltration saccharification assay, but used an earlier version of the assay (in fact, this data constituted the initial discovery of the enhanced activity of the Y245G mutant). The principal difference between the assay procedure of FIG. 3 and that of Table 3 is that different ultrafiltration membranes were used in the apparatus for the two sets of experiments. An essential feature of DSA is the retention of the macromolecular enzyme catalysts (as well as the insoluble substrate) by the membrane, while the much smaller soluble-sugar products are swept out of the reaction chamber by a buffer flux through the membrane. The ultrafiltration membrane used in the first set of experiments was an Amicon PM-10 (Amicon). With a nominal molecular-weight cut-off of 10 kDa, this membrane was considered sufficient to retain the enzyme catalysts Cel5A and Cel7A , both of which have molecular weights in excess of 40 kDa.. In fact, it was later discovered that over an extensive period of digestion such as 96 h, during which period a volume of buffer equal to more than 200 times the volume of the reaction vessel had been passed through the reaction vessel, a substantial portion of Cel5A and its Y245G variant (which are less tightly bound to the substrate than is Cel7A) was swept out of the reaction vessel and thus lost to the reaction. The result of this slow, progressive loss of catalyst meant was a drastic reduction of the reaction rate in the later part of the 96-h digestion, relative to the rate that would have been observed, had all enzyme catalyst been retained in the vessel throughout the digestion. Because all the digestions reported in Table 3 were almost shut down by 96 h of digestion, the mutant was able to retain the advantage it achieved because of its enhanced kinetics during the early part of the reaction.

Prior to the collection of the data illustrated by the progress curves of FIG. 3, the assay procedure was changed to employ a different ultrafiltration membrane, the Biomax-5 (Millipore Corporation, Bedford, Mass.), which has a nominal molecular-weight cut-off of 5 kDa. This membrane was found to provide much better retention of the enzymes, with the result that, as shown in FIG. 3, combinations of both wild-type and mutant enzymes with T. reesei cellobiohydrolase-1, were seen (even though used at only 75% of the loading of the earlier experiments and assayed at a temperature 12° C. lower) to hydrolyze a larger portion of the cellulose content of the substrate (relative to the conversion percentages reported in Table 3), and at extended digestion times were seen to approach the same extents of conversion. (Because there is a finite quantity of substrate cellulose to be converted and accessible to these pairs of enzymes, the wild-type, if given sufficient reaction time, will catch up with the mutant, even though the mutant has significantly enhanced kinetics.

Thus, although the earlier version of the assay did correctly identify the Y245G mutant as having kinetic performance superior to that of the wild-type, the later and more refined version of the assay was needed to reveal the speicific manner in which the mutant was superior (i.e., in having greatly reduced susceptibility to product inhibition.)

### EXAMPLE 5

### Structural Assessment for Y245G Enhanced Activity

The present example demonstrates the utility of the invention for identifying structural characteristics of the mutant Y245G that may be used to identify sites within other catalytic enzymes that may be modified with a similar expectation of enhanced catalytic activity over the wild-type counterpart of the particular enzymes.

Overall structural variations among wild type (Sakon et al., 1996) and Y245G are minimal. The root-mean-square deviations of Cα between wild type and Y245G is 0.22 Å. Even though the overall structures were similar, important structural changes occurred in mutant Y245G at site 246, but not at site 245. That is, compared to wild type, the torsional angle of residue 246 in the crystal structure of Y245G is shifted from 67.6° to 142.5°. In this state, the carbonyl group of Pro246 is positioned to the inside of the catalytic cleft and readily available for hydrogen bonding with water. The water molecule is not in a position in which it can form a hydrogen bond with the hydroxyl groups of Glc1. A further consequence of the torsional change at Pro246 is the retraction of Gln247 away from the enzyme cavity. In the wild type-substrate complex, Nε2 of Gln247 interacts with O2 of Glc1. Thus, one may notice that the mutation of wild type to Y245G reduces the binding energy between the leaving group and the enzyme by two means: by removing a hydrophobic platform residue and by lengthening a hydrogen bond by ˜0.5 Å. The experimental estimate for the reduction in binding energy (˜1.5 kcal/mol, see above) is supported by the density functional (DFT) calculations (see the Methods section) which yield a value of ˜3 kcal/mol.

The density functional calculations are in agreement with the assumption that the main-chain torsional angle change at Pro246 between wild type and Y245G is caused both by torsional strain and steric interactions. Torsional strain is indicated by the fact that the density function energy of Y245G calculated with the φψ-torsional angles of wild type at site 246 is ˜1.5 kcal/mol higher than that of Y245G with site 246 in the crystal structure. This finding is also in agreement with the fact that the φψ-torsional angles of wild type at Pro246 (−68.0°, 67.6°) are in a scarcely populated region of the Ramachandran plot for Pro residues, whereas the values in Y245G, at −78.7° and 142.5°, are commonly observed. The importance of steric interactions is apparent from the fact that, if wild type would adopt the ψ angle found at Pro246 in the crystal structure of Y245G, then O-Gln247 and Cδ-2-Tyr245 would be subject to a highly unfavorable interaction at ˜2.4 Å. DFT calculations indicate that the corresponding destabilization can be on the order of several tens of kcal/mol. In other proteins, a small but significant fraction of non-Gly residues have been found to adopt φψ angles that are energetically unfavorable (Karplus, 1996). The mutation of those residues in a model protein, Staphylococcal nuclease, showed that relieving such strain energy could increase the stability of the protein by 1 to 2 kcal/mol with respect to the wild type (Stites et al., 1994). The use of DFT results agreed well with the labor intensive, experimental determination of strain energy in the model protein while reducing the time and labor required. Such good results could lead to the use of DFF calculations as predictive tools in protein engineering.

To address the question as to whether the loop in Y245G had become flexible by the loss of the side chain of Tyr to Gly, we performed a detailed analysis of the temperature factors. Relative B-factor values within a molecule have been shown to contain some information about thermal atomic displacements (Kuriyan & Weis, 1991; Ringe & Petsko, 1986; Stroud & Fauman, 1995). Comparing the relative B-factor values of wild type and Y245G near their binding sites, we conclude that the mutant Gly did not significantly increase the thermal displacements. In contrast, the wild type-cellotetraose complex exhibited significantly higher temperature factors, perhaps due to the fact that cellotetraose is a true substrate, and the enzyme is in a superposition of four different states (Sakon et al., 1996).

In conclusion, the enhanced catalytic activity of the endocellulase Cel5A mutant (Y245G) is primarily due to a reduction in product inhibition. Part of the total “inhibition” that is relieved may actually reflect reversal of the depolymerization reaction by attack of bulk-solution cellobiose on the glycosyl-enzyme (i.e., transglycosylation). Nonetheless, whether the relieved inhibition is attributed to one or the other or to a combination of both of these mechanisms, it is important to note that both mechanisms involve binding of product to the enzyme active site. Thus, the central message of this study is that: (i) Theoretical binding-energy calculations utilizing high-resolution X-ray crystallographic structures of Cel5A indicated that a specific mutation (Tyr245 to Gly245) should reduce the affinity of the enzyme active site for the product, cellobiose. (ii) Initial-velocity enzyme-kinetic measurements on both the native enzyme and the mutant revealed that the affinity for cellobiose in the mutant was indeed reduced substantially when compared to the original enzyme (Kᵢ value 15.8-fold larger in the mutant). (iii) In further kinetic studies involving substantial conversion of two different insoluble cellulosic substrates (one a feasible industrial biomass feedstock) under simulated industrial process conditions, the reduced susceptibility of the engineered enzyme to cellobiose inhibition was shown, as also predicted, to translate into enhanced rates of depolymerization of cellulose. These combined results are thus a powerful confirmation of the value of an information-based approach, using structural and kinetic data to drive site-directed mutagenesis, in engineering enzymes for specific applications.

### PROPHETIC EXAMPLE 6

### Mutant Vatiants Of Y245G

It is envisioned that the information in the present disclosure that led to the creation of the specific mutant enzyme Y245G may be applied to create yet other mutant enzymes that will have an increased ability to solubilize cellulose, relative to their wild-type counterparts. For example, a number of glycohydrolases belonging to structural family 5 have been identified as being structurally analogous to EI and as having specific residues, the aromatic side chains of which may perform functions equivalent to that of Tyr-245 in EI (Table 1, left column). Mutation of these residues to the residues listed in corresponding rows of the middle column ( Trp39 of 1A3H; Trp171of 1BQC; Trp212 of 1CEN; Phe229 and/or Phe258 of 1CZ1; Trp259 and/or Trp811 of 1EDG; Trp30 of 2MAN) may reasonably be expected, on the basis of computer modeling studies, to produce a decrease in the degree of product inhibition exhibited by the resulting mutant enzymes, relative to that exhibited by the wild-type enzymes, and as a result may also be expected to exhibit improved performance in the hydrolysis of cellulose. In an analogous fashion, replacement of the residues listed in the right-hand column of Table I with residues having much less ability to form hydrogen bonds to the oxygen or hydrogen atoms of substrate hydroxyl groups can also be expected to reduce the affinity of the enzyme active site for cellobiose. The mutant enzymes that may be produced using the information in the present disclosure exemplified by, but not limited to, the examples given in Table 1.

The utility of the present invention for providing in modified form virtually any enzyme that shares with Cel5A the characteristics of being a hydrolytic depolymerizing enzyme and having a specific binding site for the leaving group, such modified form having the enhanced catalytic activity as defined herein over wild-type enzyme, is demonstrated as part of the present example.

### EXAMPLE 7

### Sequence Information

The following table provides sequence data referenced throughout the present specification.

Nucleic acid sequence for EI endoglucanase

GCGGGCGGCGGCTATTGGCACACGAGCGGCCGGGAGATCCTGGACGCGAACAACGTGCCGGTACGGA
TCGCCGGCATCAACTGGTTTGGGTTCGAAACCTGCAATTACGTCGTGCACGGTCTCTGGTCACGCGACT
ACCGCAGCATGCTCGACCAGATAAAGTCGCTCGGCTACAACACAATCCGGCTGCCGTACTCTGACGAC
ATTCTCAAGCCGGGCACCATGCCGAACAGCATCAATTTTTACCAGATGAATCAGGACCTGCAGGGTCT
GACGTCCTTGCAGGTCATGGACAAAATCGTCGCGTACGCCGGTCAGATCGGCCTGCGCATCATTCTTGA
CCGCCACCGACCGGATTGCAGCGGGCAGTCGGCGCTGTGGTACACGAGCAGCGTCTCGGAGGCTACGT
GGATTTCCGACCTGCAAGCGCTGGCGCAGCGCTACAAGGGAAACCCGACGGTCGTCGGCTTTGACTTG
CACAACGAGCCGCATGACCCGGCCTGCTGGGGCTGCGGCGATCCGAGCATCGACTGGCGATTGGCCGC
CGAGCGGGCCGGAAACGCCGTGCTCTCGGTGAATCCGAACCTGCTCATTTTCGTCGAAGGTGTGCAGA
GCTACAACGGAGACTCCTACTGGTGGGGCGGCAACCTGCAAGGAGCCGGCCAGTACCCGGTCGTGCTG
AACGTGCCGAACCGCCTGGTGTACTCGGCGCACGACTACGCGACGAGCGTCTACCCGCAGACGTGGTT
CAGCGATCCGACCTTCCCCAACAACATGCCCGGCATCTGGAACAAGAACTGGGGATACCTCTTCAATC
AGAACATTGCACCGGTATGGCTGGGCGAATTCGGTACGACACTGCAATCCACGACCGACCAGACGTTGG
CTGAAGACGCTCGTCCAGTACCTACGGCCGACCGCGCAATACGGTGCGGACAGCTTCCAGTGGACCTT
CTGGTCCTGGAACCCCGATTCCGGCGACACAGGAGGAATTCTCAAGGATGACTGGCAGACGGTCGACA
CAGTAAAAGACGGCTATCTCGCGCCGATCAAGTCGTCGATTTTCGATCCTGTCTAATGAATCGCCTAGC
AGTCAACCGTCCCCGTCGGTGTCGCCGTCTCCGTCGCCGAGCCCGTCGGCGAGTCGGACGCCGACGCC
TACTCCGACGCCGACAGCCAGCCCGACGCCAACGCTGACCCCTACTGCTACGCCCACGCCCACGGCAA
GCCCGACGCCGTCACCGACGGCAGCCTCCGGAGCCCGCTGCACCGCGAGTTACCAGGTCAACAGCGAT
TGGGGCAATGGCTTCACGGTAACGGTGGCCGTGACAAATTCCG

Amino acid sequence for EI endoglucanse

AGGGYWHTSGREILDANNVPVRIAGINWFGFETCNYVVHGLWSRDYRSMLDQIKSLGYNTIRLPYSDDILK
PGTMPNSINFYQMNQDLQGLTSLQVMDKIVAYAGQIGLRIILDRHRPDCSGQSALWYTSSVSEATWISDLQ
ALAQRYKGNPTVVGFDLHNEPHDPACWGCGDPSIDWRLAAERAGNAVLSVNPNLLIFVEGVQSYNGDSY
WWGGNLQGAGQYPVVLNVPNRLVYSAHDYATSVYPQTWFSDPTFPNNMPGIWNKNWGYLFNQNIAPVW
LGEFGTTLQSTTDQTWLKTLVQYLRPTAQYGADSFQWTFWSWNPDSGDTGGILKDDWQTVDTVKDGYLA
PIKSSIFDPVG

DNA sequence for Y245G Mutant with mutation site underlined.

GCGGGCGGCGGCTATTGGCACACGAGCGGCCGGGAGATCCTGGACGCGAACAACGTGCCGGTACGGA
TCGCCGGCATCAACTGGTTTGGGTTCGAAACCTGCAATTACGTCGTGCACGGTCTCTGGTCACGCGACT
ACCGCAGCATGCTCGACCAGATAAAGTCGCTCGGCTACAACACAATCCGGCTGCCGTACTCTGACGAC
ATTCTCAAGCCGGGCACCATGCCGAACAGCATCAATTTTTACCAGATGAATCAGGACCTGCAGGGTCT
GACGTCCTTGCAGGTCATGGACAAAATCGTCGCGTACGCCGGTCAGATCGGCCTGCGCATCATTCTTGA
CCGCCACCGACCGGATTGCAGCGGGCAGTCGGCGCTGTGGTACACGAGCAGCGTCTCGGAGGCTACGT
GGATTTCCGACCTGCAAGCGCTGGCGCAGCGCTACAAGGGAAACCCGACGGTCGTCGGCTTTGACTTG
CACAACGAGCCGCATGACCCGGCCTGCTGGGGCTGCGGCGATCCGAGCATCGACTGGCGATTGGCCGC
CGAGCGGGCCGGAAACGCCGTGCTCTCGGTGAATCCGAACCTGCTCATTTTCGTCGAAGGTGTGCAGA
GCTACAACGGAGACTCCTACTGGTGGGGCGGCAACCTGCAAGGAGCCGGCCAGTACCCGGTCGTGCTG
AACGTGCCGAACCGCCTGGTGTACTCGGCGCACGACTACGCGACGAGCGTCGGCCCGCAGACGTGGTT
CAGCGATCCGACCTTCCCCAACAACATGCCCGGCATCTGGAACAAGAACTGGGGATACCTCTTCAATC
AGAACATTGCACCGGTATGGCTGGGCGAATTCGGTACGACACTGCAATCCACGACCGACCAGACGTGG
CTGAAGACGCTCGTCCAGTACCTACGGCCGACCGCGCAATACGGTGCGGACAGCTTCCAGTGGACCTT
CTGGTCCTGGAACCCCGATTCCGGCGACACAGGAGGAATTCTCAAGGATGACTGGCAGACGGTCGACA
CAGTAAAAGACGGCTATCTCGCGCCGATCAAGTCGTCGATTTTCGATCCTGTCTAATGAATCGCCTAGC
AGTCAACCGTCCCCGTCGGTGTCGCCGTCTCCGTCGCCGAGCCCGTCGGCGAGTCGGACGCCGACGCC
TACTCCGACGCCGACAGCCAGCCCGACGCCAACGCTGACCCCTACTGCTACGCCCACGCCCACGGCAA
GCCCGACGCCGTCACCGACGGCAGCCTCCGGAGCCCGCTGCACCGCGAGTTACCAGGTCAACAGCGATTGGGGCAAT

Translated amino acid sequence for Y245G mutation, with modification underlined.

AGGGYWHTSGREILDANNVPVRIAGINWFGFETCNYVVHGLWSRDYRSMLDQIKSLGYNTIRLPYSDDILK
PGTMPNSINFYQMNQDLQGLTSLQVMDKIVAYAGQIGLRIILDRHRPDCSGQSALWYTSSVSEATWISDLQ
ALAQRYKGNPTVVGFDLHNEPHDPACWGCGDPSIDWRLAAERAGNAVLSVNPNLLIFVEGVQSYNGDSY
WWGGNLQGAGQYPVVLNVPNRLVYSAHDYATSVGPQTWFSDPTFPNNMPGIWNKNWGYLFNQNIAPVW
LGEFGTTLQSTTDQTWLKTLVQYLRPTAQYGADSFQWTFWSWNPDSGDTGGILKDDWQTVDTVKDGYLA
PIKSSIFDPV

DNA sequence for W42R Mutant with mutation site underlined

GCGGGCGGCGGCTATTGGCACACGAGCGGCCGGGAGATCCTGGACGCGAACAACGTGCCGGTACGGA
TCGCCGGCATCAACTGGTTTGGGTTCGAAACCTGCAATTACGTCGTGCACGGTCTCCGGTCACGCGACT
ACCGCAGCATGCTCGACCAGATAAAGTCGCTCGGCTACAACACAATCCGGCTGCCGTACTCTGACGAC
ATTCTCAAGCCGGGCACCATGCCGAACAGCATCAATTTTTACCAGATGAATCAGGACCTGCAGGGTCT
GACGTCCTTGCAGGTCATGGACAAAATCGTCGCGTACGCCGGTCAGATCGGCCTGCGCATCATTCTTGA
CCGCCACCGACCGGATTGCAGCGGGCAGTCGGCGCTGTGGTACACGAGCAGCGTCTCGGAGGCTACGT
GGATTTCCGACCTGCAAGCGCTGGCGCAGCGCTACAAGGGAAACCCGACGGTCGTCGGCTTTGACTTG
CACAACGAGCCGCATGACCCGGCCTGCTGGGGCTGCGGCGATCCGAGCATCGACTGGCGATTGGCCGC
CGAGCGGGCCGGAAACGCCGTGCTCTCGGTGAATCCGAACCTGCTCATTTTCGTCGAAGGTGTGCAGA
GCTACAACGGAGACTCCTACTGGTGGGGCGGCAACCTGCAAGGAGCCGGCCAGTACCCGGTCGTGCTG
AACGTGCCGAACCGCCTGGTGTACTCGGCGCACGACTACGCGACGAGCGTCTACCCGCAGACGTGGTT
CAGCGATCCGACCTTCCCCAACAACATGCCCGGCATCTGGAACAAGAACTGGGGATACCTCTTCAATC
AGAACATTGCACCGGTATGGCTGGGCGAATTCGGTACGACACTGCAATCCACGACCGACCAGACGTGG
CTGAAGACGCTCGTCCAGTACCTACGGCCGACCGCGCAATACGGTGCGGACAGCTTCCAGTGGACCTT
CTGGTCCTGGAACCCCGATTCCGGCGACACAGGAGGAATTCTCAAGGATGACTGGCAGACGGTCGACA
CAGTAAAAGACGGCTATCTCGCGCCGATCAAGTCGTCGATTTTCGATCCTGTCTAATGAATCGCCTAGC
AGTCAACCGTCCCCGTCGGTGTCGCCGTCTCCGTCGCCGAGCCCGTCGGCGAGTCGGACGCCGACGCC
TACTCCGACGCCGACAGCCAGCCCGACGCCAACGCTGACCCCTACTGCTACGCCCACGCCCACGGCAA
GCCCGACGCCGTCACCGACGGCAGCCTCCGGAGCCCGCTGCACCGCGAGTTACCAGGTCAACAGCGAT
TGGGGCAATGGCTTCACGGTAACGGTGGCCGTGACAAATTCCG

Translated amino acid sequence for W42R mutation, with modification underlined.

AGGGYWHTSGREILDANNVPVRIAGINWFGFETCNYVVHGLRSRDYRSMLDQIKSLGYNTIRLPYSDDILKP
GTMPNSINFYQMNQDLQGLTSLQVMDKIVAYAGQIGLRIILDRHRPDCSGQSALWYTSSVSEATWISDLQA
LAQRYKGNPTVVGFDLHNEPHDPACWGCGDPSIDWRLAAERAGNAVLSVNPNLLIFVEGVQSYNGDSYW
WGGNLQGAGQYPVVLNVPNRLVYSAHDYATSVYPQTWFSDPTFPNNMPGIWNKNWGYLFNQNIAPVWL
GEFGTTLQSTTDQTWLKTLVQYLRPTAQYGADSFQWTFWSWNPDSGDTGGILKDDWQTVDTVKDGYLAP
IKSSIFDPV

DNA sequence for Y82R Mutant with mutation site underlined.

GCGGGCGGCGGCTATTGGCACACGAGCGGCCGGGAGATCCTGGACGCGAACAACGTGCCGGTACGGA
TCGCCGGCATCAACTGGTTTGGGTTCGAAACCTGCAATTACGTCGTGCACGGTCTCTGGTCACGCGACT
ACCGCAGCATGCTCGACCAGATAAAGTCGCTCGGCTACAACACAATCCGGCTGCCGTACTCTGACGAC
ATTCTCAAGCCGGGCACCATGCCGAACAGCATCAATTTTCGGCAGATGAATCAGGACCTGCAGGGTCT
GACGTCCTTGCAGGTCATGGACAAAATCGTCGCGTACGCCGGTCAGATCGGCCTGCGCATCATTCTTGA
CCGCCACCGACCGGATTGCAGCGGGCAGTCGGCGCTGTGGTACACGAGCAGCGTCTCGGAGGCTACGT
GGATTTCCGACCTGCAAGCGCTGGCGCAGCGCTACAAGGGAAACCCGACGGTCGTCGGCTTTGACTTG
CACAACGAGCCGCATGACCCGGCCTGCTGGGGCTGCGGCGATCCGAGCATCGACTGGCGATTGGCCGC
CGAGCGGGCCGGAAACGCCGTGCTCTCGGTGAATCCGAACCTGCTCATTTTCGTCGAAGGTGTGCAGA
GCTACAACGGAGACTCCTACTGGTGGGGCGGCAACCTGCAAGGAGCCGGCCAGTACCCGGTCGTGCTG
AACGTGCCGAACCGCCTGGTGTACTCGGCGCACGACTACGCGACGAGCGTCTACCCGCAGACGTGGTT
CAGCGATCCGACCTTCCCCAACAACATGCCCGGCATCTGGAACAAGAACTGGGGATACCTCTTCAATC
AGAACATTGCACCGGTATGGCTGGGCGAATTCGGTACGACACTGCAATCCACGACCGACCAGACGTGG
CTGAAGACGCTCGTCCAGTACCTACGGCCGACCGCGCAATACGGTGCGGACAGCTTCCAGTGGACCTT
CTGGTCCTGGAACCCCGATTCCGGCGACACAGGAGGAATTCTCAAGGATGACTGGCAGACGGTCGACA
CAGTAAAAGACGGCTATCTCGCGCCGATCAAGTCGTCGATTTTCGATCCTGTCTAATGAATCGCCTAGC
AGTCAACCGTCCCCGTCGGTGTCGCCGTCTCCGTCGCCGAGCCCGTCGGCGAGTCGGACGCCGACGCC
TACTCCGACGCCGACAGCCAGCCCGACGCCAACGCTGACCCCTACTGCTACGCCCACGCCCACGGCAA
GCCCGACGCCGTCACCGACGGCAGCCTCCGGAGCCCGCTGCACCGCGAGTTACCAGGTCAACAGCGAT
TGGGGCAATGGCTTCACGGTAACGGTGGCCGTGACAAATTCCG

Translated amino acid sequence for Y82R mutation, with modification underlined.

AGGGYWHTSGREILDANNVPVRIAGINWFGFETCNYVVHGLWSRDYRSMLDQIKSLGYNTIRLPYSDDILK
PGTMPNSINFRQMNQDLQGLTSLQVMDKIVAYAGQIGLRIILDRHRPDCSGQSALWYTSSVSEATWISDLQA
LAQRYKGNPTVVGFDLHNEPHDPACWGCGDPSIDWRLAAERAGNAVLSVNPNLLIFVEGVQSYNGDSYW
WGGNLQGAGQYPVVLNVPNRLVYSAHDYATSVYPQTWFSDPTFPNNMPGIWNKNWGYLFNQNIAPVWL
GEFGTTLQSTTDQTWLKTLVQYLRPTAQYGADSFQWTFWSWNPDSGDTGGILKDDWQTVDTVKDGYLAP
IKSSIFDPV

### BIBLIOGRAPHY

The following references are specifically incorporated herein by reference in their entirety.

U.S. Pat. No.: 5,536,655

Baker, J. O., Adney, W. S., Thomas, S. R., Nieves, R. A., Chou, Y. -C., Vinzant, T. B., Tucker, M. P., Laymon, R. A., & Himmel, M. E. (1995) in Enzymatic degradation of insoluble carbohydrates Vol. 618, pp 113-141, (Saddler, J. N. & Penner, M. H., Eds.) American Chemical Society, Washington, DC.

Baker, J. O., Vinzant, T. B., Ehrman, C. I., Adney, W. S., & Himmel, M. E. (1997) Appl. Biochem. Biotech. 63-65, 585-595.

Baker, J. O., Ehrman, C. I., Adney, W. S., Thomas, S. R., & Himmel, M. E. (1998) Appl. Biochem. Biotechnol. 70, 395-403.

Becke, A. D. (1993) J. Chem. Phys. 98, 5648-5652. Bergeron, P. (1996) in Handbook on bioethanol (Wyman, C. E., Ed.) pp 179-195, Taylor & Francis, Washington, DC.

Diederichs, K., & Karplus, P. A. (1997) Nature Struct. Biol. 4, 269-275.

Divne, C., Stahlberg, J., Reinikainen, T., Ruohonen, L., Pettersson, G., Knowles, J. K. C., Teeri, T. T., & Jones, A. (1994) Science 265, 524-528.

Frisch, M. J. et al. (1995) Gaussian 94, Gaussian, Inc., Pittsburgh, Pa. Himmel, M. E., Adney, W. S., Baker, J. O., Elander, R., McMillan, J. D., Nieves, R. A., Sheehan, J. J., Thomas, S. R., Vinzant, T. B., & Zhang, M. (1997) in Fuels and chemicals from biomass (Saha, B. & Woodward, J., Eds.) Vol. 666, pp 2-45, American Chemical Society, Washington, DC.

Himmel, M. E., Adney, W. S., Grohmann, K., & Tucker, M. P. (1994) U.S. Pat. No. 5,275,944, January 4.

Hsu, T. -A. (1996) in Handbook on bioethanol (Wyman, C. E., Ed.) pp 179-195, Taylor & Francis, Washington, DC.

Irwin, D. C., Spezio, M., Walker, L. P., & Wilson, D. B. (1993) Biotechnol Bioeng. 42, 1002-1013.

Karplus, P. A. (1996) Protein Sci. 5, 1406-1420.

Koshland, D. E. J. (1953) Biol. Rev. 28, 416-436.

Krishnan, R., Binkley, J. S., Seeger, R., & Pople, J. A. (1980) J. Chem. Phys. 72, 650-654.

Kuriyan, J., & Weis, W. I. (1991) Proc. Natl. Acad. Sci. USA 88, 2773-2777.

Laskowski, R. A., MacArthur, M. W., Moss, D. S., & Thornton, J. M. (1993) J. Appl. Crystallogr. 26, 283-291.

McCarter, J. D., & Withers, S. G. (1994) Curr. Opin. Struct. Biol. 4, 885-892.

Moreau, A., Shareck, F., Kluepfel, D., & Morosoli, R. (1994) Eur. J. Biochem. 219, 261-266.

Nidetzky, B., Steiner, W., & Claeyssens, M. (1994) Biochem. J. 303, 817-823.

Otwinowski, Z. (1993) in Proceedings of the CCP4 study weekend: data collection and processing, pp 29-30, Warrington, UK.

Parr, R. G., & Yang, W. (1989) Density-functional theory of atoms and molecules, Oxford University Press, Oxford, UK.

Pearson, E. S., & Hartley, H. O. (1970) Biometrika Tables for Statisticians, Cambridge University Press, Cambridge, UK.

Ramek, M., Momany, F. A., Miller, D. M., & Schäfer, L. (1996) J. Mol. Struct. 375, 189-191.

Ringe, D., & Petsko, G. A. (1986) Methods Enzymol. 131, 389-433.

Rouvinen, J., Bergfors, T., Teeri, T., Knowles, J. K. C., & Jones, T. A. (1990) Science 249, 380-386.

Sakon, J., Adney, W. S., Himmel, M. E., Thomas, S. R., & Karplus, P. A. (1996) Biochemistry 35, 10648-10660.

Sakon, J., Irwin, D., Wilson, D. B., & Karplus, P. A. (1997) Nature Struct. Biol. 4, 810-818.

Segel, I. H. (1975) Enzyme Kinetics, pp 109-111, John Wiley & Sons, New York, N.Y.

Sheehan, J. J. (1994) in Enzymatic conversion of biomass for fuels production (Himmel, M. E., Baker, J. O., & Overend, R. P., Eds.) Vol. 566, pp 1-52, American Chemical Society, Washington, DC.

Sheldrick, G. M. (1990) Acta Crystallogr. Sect. A 46, 467-473.

Sinnott, M. L. (1990) Chem. Rev. 90, 1171-1202.

Snedecor, G. W., & Cochran, W. G. (1967) Statistical Methods Iowa State University Press, Ames, Iowa.

Spezio, M., Wilson, D. B., & Karplus, P. A. (1993) Biochemistry 32, 9906-9916.

Stites, W. E., Meeker, A. K., & Shortle, D. (1994) J. Mol. Biol. 235, 27-32.

Stroud, R. M., & Fauman, E. B. (1995) Protein Sci. 4, 2392-2404.

Thomas, S. R., Laymon, R. A., Chou, Y. -C., Tucker, M. P., Vinzant, T. B., Adney, W. S., Baker, J. O., Nieves, R. A., Mielenz, J. R., & Himmel, M. E. (1995) in Enzymatic Degradation of Insoluble Polysaccharides, (Saddler, J. N., & Penner, M. H., Eds.) Vol. 618, pp 208-236, American Chemnical Society, Washington, DC.

Vriend, G. (1990) J. Mol. Graph. 8, 52-56.

Wyman, C. E., Bain, R. L., Hinman, N. D., & Stevens, D. J. (1993) in Renewable energy: sources for fuels and electricity (Johansson, T. B., Kelly, H., Reddy, A. K. N., & Williams, R. H., Eds.) Island Press, Washington, D.C.

What is claimed is:

1. A method for increasing the specific activity of a glycosyl hydrolase on a substrate, comprising replacing a hydrophobic surface binding amino acid of the hydrolase with a positively charged amino acid, to provide a mutant glycosyl hydrolase,

2. The method of claim 1, wherein the hydrophobic surface binding amino acid includes tryptophan or tyrosine and the positively charged amino acid is arginine.

3. A method for increasing the specific activity of a glycosyl hydrolase on a substrate, comprising replacing an active site associated glycosyl-stabilizing amino acid of the hydrolase with an amino acid, the replacing amino acid not strongly retarding cellobiose from leaving the active site to provide a mutant glycosyl hydrolase.

4. The method of claim 3, wherein the glycosyl-stabilizing amino acid comprises tyrosine 3 and the replacing amino acid comprises glycine.

5. The methods of claims 1 and 3 wherein replacing comprises site-directed-mutagenesis.

6. The methods of claims 1 and 3 wherein the mutant glycosyl hydrolase comprises a mutant EI endoglucanase.

7. The methods of claims 1 and 3 wherein the mutant glycosyl hydrolase comprises Y245G, Y42R, W82R, or a mixture thereof.

8. The methods of claims 1 and 3, wherein the substrate comprises pretreated biomass.

9. A mutant glycosyl hydrolase having enhanced catalytic activity, said mutant glycosyl hydrolase comprising an amino having a positively charged amino acid at a position occupied by a hydrophobic surface binding amino acid in a wild-type glycosyl hydrolase amino acid sequence, wherein said mutant glycosyl hydrolase has an enhanced catalytic activity of 10% to 50% compared to catalytic activity of the wild-type glycosyl hydrolase.

10. The mutant glycosyl hydrolase of claim 9 further defined as a cellulase.

11. The mutantglycosyl hydrolase of claim 9 further defined as Y245G.

12. The mutant glycosly hydrolase of claim 9 further defined as Y42R.

13. The mutant glycosyl hydrolase of claim 9 further defined as a mannanase.

14. The mutant glycosyl hydrolase of claim 9 further defined as comprising W82R.

15 A method for converting a biomass into ethanol comprising:
a. mixing a composition comprising biomass with a mutant glycosyl hydrolase having enhanced catalytic activity over a wild-type glycosyl hydrolase to provide a soluble fermentable sugar preparation; and
b. fermenting said soluble fermentable sugar preparation to provide a composition comprising ethanol.

16. The method of claim 15 wherein the biomass is a cellulosic biomass.

17. The method of claim 15 wherein the mutant glycosyl hydrolase is Y245G.

18. The method of claim 15 wherein the mutant glycosyl hydrolase is Y82R.

19. The method of claim 15 wherein the mutant glycosyl hydrolase is W42R.

20. The method of claim 15 wherein the mutant glycosyl hydrolase comprises Y245G, Y82R, or W42R.

21. The method of claim 15 wherein the biomass is further admixed with a glycohydrolase.

22. A method for converting a biomass to a lactic acid comprising:
a. admixing a composition comprising a biomass with a glycosyl mutant hydrolase having enhanced catalytic activity over a wild-type glycosyl hydrolase to provide a soluble fermentable sugar preparation; and
b. fermenting said soluble fermentable sugar preparation to provide lactic acid.

23. The method of claim 22 wherein the lactic acid is further defined as a monomer feedstock for production of biodegradable plastics.

24. The method of claim 22 wherein the mutant glycosyl hydrolase is Y82R, W42R, Y245G, or a mixture thereof.

25. The method of claim 22 wherein the glycosyl hydrolase mutant is mutant Y82R, W42R, Y245G, or a mixture thereof.

26. A method for increasing the specific activity of a hydrolytic depolymerizing enzyme, comprising replacing an extended-active site residue that binds strongly to the leaving group with another that binds much less strongly to the leaving group.
